(19) United States
(12) Patent Application Publication
(10) Pub. No.: US 2003/0212400 A1
(43) Pub. Date: Nov. 13, 2003

(54) Methods for treating spinal stenosis by pedicle distraction

(75) Inventors: Wilhelm Bloemer, Unteruhldingen-Muehlhofen (DE); Thierry Marnay, Montpellier (FR)

Correspondence Address: LAW OFFICE OF BARRY R LIPSITZ, 755 MAIN STREET, MONROE, CT, 06468, US

(73) Assignee: AESCULAP AG & Co. KG, Tuttlingen (DE)

(21) Appl. No.: 10/386,357

(22) Filed: Mar. 11, 2003

Related U.S. Application Data
(60) Provisional application No. 60/363,854, filed on Mar. 12, 2002.

(51) Int. Cl. A61B 17/56

(52) U.S. Cl. 606/61

(57) ABSTRACT

The invention provides methods for treating spinal stenosis by pedicle distraction. The vertebral segment is cut so that the pedicles are separated from the main portion of the vertebral segment. After being cut, each pedicle is separated from the main portion and fixed at a distance from the main portion using screws. For example, two-component compression screws or stenosis screws may be used to fix the pedicles from the main portion. The screws may be inserted through bores drilled into the vertebral segment. The bores are preferably drilled prior to the cutting. As a result of fixing the cut portion at a distance from the main portion, there will be a gap between the main portion and the two pedicles, thereby enlarging the cross-section of the spinal column.

This application claims the benefit of U.S. Provisional Application No. 60/363,854, filed Mar. 12, 2002, the entire disclosure of which is hereby incorporated by reference.

### BACKGROUND OF THE INVENTION

The present invention relates generally to correcting spinal defects. More specifically, the present invention relates to methods for alleviating spinal stenosis by enlarging the cross-section of the spinal canal in a vertebral or spinal segment by pedicle distraction.

If the cross section of the spinal canal is too small, a condition called “lumbar spinal stenosis” exists and results in trauma to the nerves running through the spinal canal. Such stenosis can be due to degenerative effects of the vertebral material.

To date, surgeons have tried a variety of ways to broaden the spinal canal. However, such prior art procedures may be very dangerous, as the neural material may be traumatized.

It would be advantageous to provide methods for alleviating spinal stenosis that do not damage the neural material. It would be further advantageous to provide for enlargement of the cross-section of the spinal canal by distraction of the pedicles.

The methods of the present invention provide the foregoing and other advantages.

### SUMMARY OF THE INVENTION

The present invention relates to methods for alleviating spinal stenosis by enlarging the cross-section of the spinal canal in a vertebral or spinal segment by distraction of the pedicles.

The inventive method comprises cutting the vertebral or spinal segment so that the pedicles of the vertebral segment are separated from the main portion of the vertebral segment. After being cut, the pedicle portion of the vertebral segment is separated from the main portion and fixed at a distance from the main portion using screws. For example, two-component compression screws or stenosis screws may be used to fix the pedicles from the main portion. The screws may be inserted through bores drilled into the vertebral segment. The bores are preferably drilled prior to the cutting. As a result of fixing the cut portion at a distance from the main portion, there will be a gap between the main portion and the two pedicles, thereby enlarging the cross-section of the spinal column.

### BRIEF DESCRIPTION OF THE DRAWINGS

The present invention will hereinafter be described in conjunction with the appended drawing figures, wherein like numerals denote like elements, and:

FIG. 1 shows a cross-section of a vertebral segment in accordance with an example embodiment of the invention;

FIG. 2 shows a cross-section of a vertebral segment in which pedicles are separated from the main portion of the vertebral segment according to the invention;

FIG. 3 shows a cross-section of a vertebral segment in which one pedicle is separated from the main portion of the vertebral segment according to the invention;

FIG. 4 shows an example embodiment of a two-component compression screw which may be used in accordance with an example embodiment of the invention;

FIG. 5 shows an example embodiment of a stenosis screw which may be used in accordance with an example embodiment of the invention; and

FIG. 6 shows a cross-section of a vertebral segment in accordance with an example embodiment of the invention where the stenosis screws of FIG. 5 are used.

### DETAILED DESCRIPTION OF THE INVENTION

The ensuing detailed description provides exemplary embodiments only, and is not intended to limit the scope, applicability, or configuration of the invention. Rather, the ensuing detailed description of the exemplary embodiments will provide those skilled in the art with an enabling description for implementing the invention. It should be understood that various changes may be made in the function and arrangement of elements without departing from the spirit and scope of the invention as set forth in the appended claims.

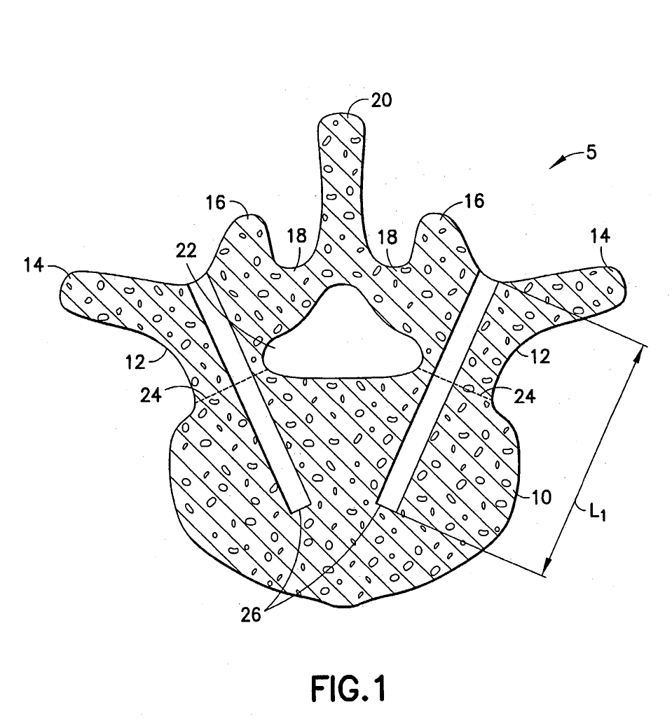

Example methods for treating spinal stenosis are shown in FIGS. 1-3. FIG. 1 shows the relevant details of a cross-section of a vertebral or spinal segment generally designated 5. The vertebral segment 5 comprises a main segment 10 which is roughly oval in cross section, two pedicles 12 above the main portion 10, transverse processes 14 extending from the pedicles 12, superior articular processes 16, laminae 18, a spinous process 20, and a spinal cord 22.

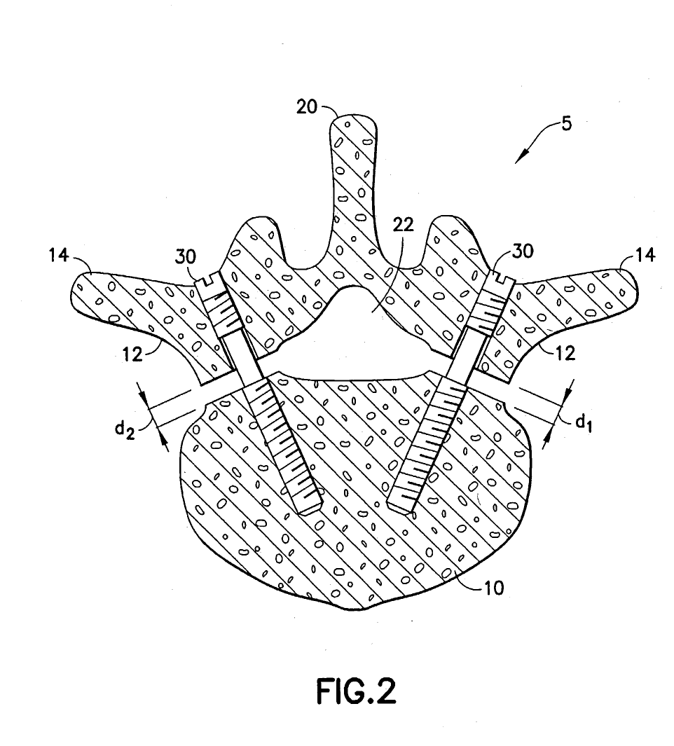

In accordance with an example embodiment of the invention, the vertebral segment 5 is cut to separate both of the pedicles 12 from a main portion 10 of the vertebral segment 5. The position of the cuts is shown (approximately) in FIG. 1 as dashed lines 24. After being cut, both of the pedicles 12 are separated from the main segment 10 and then fixed at first and second respective distances (d₁, d₂ of FIG. 2) from the main segment 10. Each of the separated pedicles 12 may be fixed at the respective distances d₁, d₂ from the main segment using a screw 30 as shown in FIG. 2.

As shown in FIG. 1, a first bore 26 may be drilled a first length L, through each of the pedicles 12 and into the main segment 10 before cutting of the pedicles from the main segment 10. The screw 30 may be inserted through each of the first bores 26 to separate and fix the pedicles 12 at the respective distances d₁ and d₂ from the main segment 10 after the segment is cut, as shown in FIG. 2. It should be appreciated that although the bores 26 are shown as having equal lengths in FIG. 1, they can alternatively have different lengths.

As a result of fixing the cut portion at a distance from the main portion 10, there will be respective gaps d₁, d₂ created between the main portion 10 and the two pedicles 12. Through these gaps, which extend into the spinal canal 22, the cross section of the spinal canal 22 will be enlarged.

Each of the first and second distances d₁, d₂ may be, e.g., approximately 3 to 5 mm. The first and second distances d₁, d₂ may be approximately equal. Alternatively, the first and second distances may differ depending on the desired outcome of the procedure.

In an alternate example embodiment, the pedicle portions 12 may be cut from the main portion 10 prior to the drilling of the bores 26 into the pedicles 12 and the main portion 10.

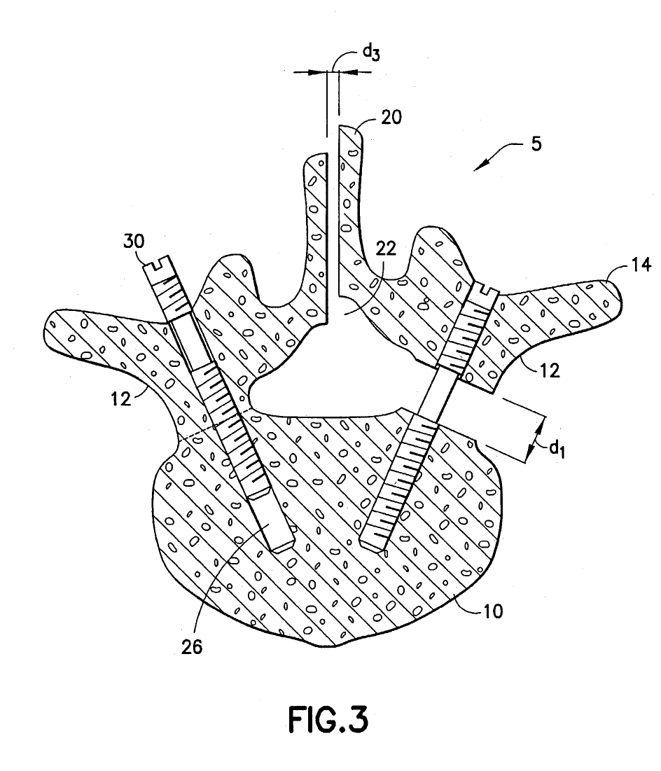

The screw 30 used to fix one of the pedicles 12 may be arranged approximately in parallel to the screw 30 used to fix the other pedicle 12 to provide for a parallel shifting of the pedicles 12 during separation. In FIGS. 2 and 3, the two screws 30 are shown as angled with respect to each other. The screws should be inserted at a small angle (i.e. close to parallel with one another) in order to enable a parallel shifting of the two pedicles during the separation operation. It should be appreciated that the bone material of the vertebral segment 5 is flexible enough to allow for limited movement to accommodate the separating of the pedicles 12 when the screws are inserted at a small angle with respect to one another.

In an example embodiment of the invention, a drill may be inserted through a small epidermal incision in the area of the vertebral segment 5 for drilling of the bores 26 into the vertebral segment 5. A cutting device may be inserted into the bore 26 for cutting of the vertebral segment 5. The cuts 24 separating the pedicle 12 from the main portion 10 may be made using a saw or a shaver-like instrument. The cuts 24 may be made from the outside of the vertebral segment 5, using a wire saw, such as the Olivecrona and Gigli wire saws sold by Aesculap AG & Co. KG. Alternatively, the cuts 24 may be made using an oscillating blade saw, a chisel, or other cutting devices known in the art.

One pedicle 12 may be separated and fixed at the first respective distance d₁ before the other pedicle 12 is separated and fixed at the second respective distance d₂. The left hand side of FIG. 3 shows a pedicle 12 prior to separation and the right hand side of FIG. 3 shows the pedicle 12 after being cut and separated from the main portion 10.

In a further example embodiment of the invention as shown in FIG. 3, the vertebral segment 5 may also be cut in a longitudinal direction between the two pedicles 12 (i.e. along the center of the spinous process 20), separating the pedicles 12 from each other in order create a gap d₃ between the two pedicles 12. The gap may be approximately 1 mm, for example. This cut is optional and will normally not be made. Such an additional longitudinal cut between the two pedicles 12 may be necessary in special cases requiring additional enlargement of the spinal canal 22.

The screw 30 used to fix the pedicles 12 which are cut from the main portion 10 and to maintain a gap d₁, d₂ between the pedicles 12 and the main portion 10 may be special kind of bone screw. These bone screws may be screws which are normally used as compression screws to compress two bone segments, but which can also be used as distraction screws.

The screw 30 may be, for example, a compression screw as shown in UK patent application no. GB 2,323,533 entitled “Orthopaedic Modular Compression Screw”, filed on Mar. 25, 1997 and published on Sep. 30, 1998, which is incorporated herein and made a part hereof by reference.

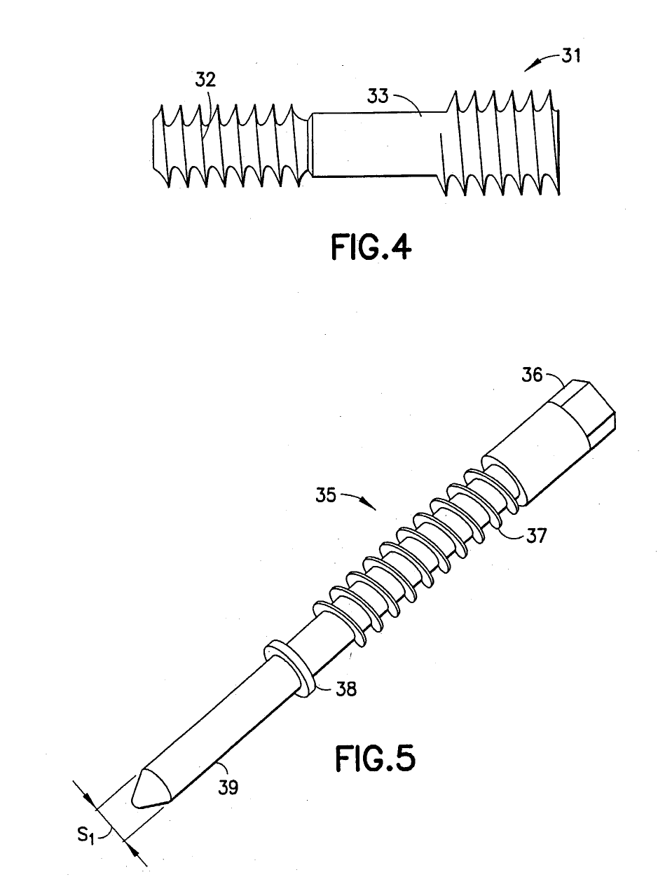

FIG. 4 shows such a compression screw 31 consisting of two components 32 and 33 having coaxial external threads. Each component 32, 33 can be rotated separately from the other component and is fixed in an axial direction. The screw 31 shown in FIG. 4 is described in detail in the aforementioned UK patent application. Two-component screws 31 of the type disclosed in the aforementioned UK patent application would be particularly effective for the separation because these screws 31 do not require much space and can be introduced along their axis through the bores 26.

An example embodiment using the screw 31 is shown in FIGS. 2 and 3. After the cut 24 has been made separating the pedicle 12 from the main portion 10, one of the components 32 of the screw 31 is screwed into the bore 26 in the main portion 10 and the other component 33 is only partly screwed into the bore 26 in the pedicle 12 while the pedicle 12 is still positioned close to the main portion 10. If the component 33 which is screwed into the pedicle 12 is further rotated on the other screw component 32 which is already secured in the main portion 10, this will separate the pedicle from the main portion such as shown on the right hand side of FIG. 3.

It would also be possible to use component screws 31 where each component 32, 33 of the screw 31 may have threads with different pitches.

In an alternate example embodiment as shown in FIG. 5, the screws 30 may each comprise a stenosis screw 35. The stenosis screw 35 may comprise a head portion 36 adapted to accept a turning tool for rotating the screw 35, a threaded portion 37 adjacent the head portion 36, a shoulder portion 38 adjacent the threaded portion 37, and a non-threaded pin portion 39 adjacent the shoulder portion 38.

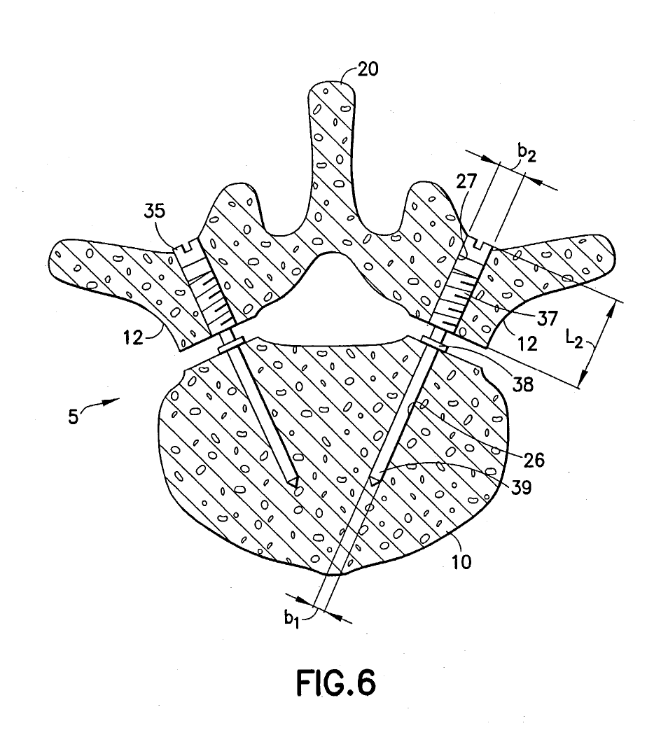

An example embodiment where such a stenosis screw 35 is used is shown in FIG. 6. In such an embodiment, the diameter b₁ of the first bore 26 may correspond to a diameter s₁ of the non-threaded pin portion 39 of the stenosis screw 35. A second bore 27 may be drilled over the first bore 26. The second bore 27 may have a larger diameter b₂ than the diameter b₁ of the first bore and a second length L₂ which is shorter than the first length L₁ of the first bore 26. Such over drilling of the first bore 26 results in a two-section bore having a first upper section (defined by length L₂) in the pedicle 12 having a larger diameter b₂ than a second lower section (defined by length L₁-L₂) in the main segment 10. The stenosis screw 35 may be inserted into the two-section bore such that the unthreaded pin portion 39 penetrates into the lower section of the two-section bore. The threaded portion 37 will penetrate into the bone material of the pedicle 12 surrounding the upper section of the two-section bore. The shoulder 38 will abut the main segment 10 and limit the penetration of the unthreaded pin portion 39 into the lower section. Once the screw 35 is inserted, rotating of the screw 35 causes the pedicle 12 to separate from the main segment 10.

The diameter b, of the lower section of the two-section bore may be approximately 2.7 mm. The diameter b₂ of the upper section of the two-section bore may be approximately 4.0 mm. The first length L₁ may be approximately 35 mm. The second length L₂ may be approximately 15 mm.

The overall shape of the stenosis screw 35 may be cylindrical (as shown in FIG. 5). Alternatively, the stenosis screw 35 may be conical in shape.

Those skilled in the art will appreciate that a variety of different screw types or other fixing devices could be used for holding the pedicles in the separated position.

It should now be appreciated that the present invention provides advantageous methods for treating spinal stenosis by distraction of the pedicles to enlarge the spinal canal, without traumatizing the neural material.

Although the invention has been described in connection with various illustrated embodiments, numerous modifications and adaptations may be made thereto without departing from the spirit and scope of the invention as set forth in the claims.

What is claimed is:

1. A method for treating spinal stenosis, comprising:
cutting a vertebral segment having two pedicles to separate both of said pedicles from a main portion of the segment; and
fixing both of said separated pedicles at first and second respective distances from the main segment.

2. A method in accordance with claim 1, wherein each of said separated pedicles is fixed at said respective distance from the main segment using a screw.

3. A method in accordance with claim 2, wherein:
the screw used to fix one of the pedicles is arranged approximately in parallel to the screw used to fix the other pedicle to provide for a parallel shifting of the pedicles during separation.

4. A method in accordance with claim 1, further comprising:
drilling a first bore a first length through each of the pedicles and into the main segment before cutting of said pedicles from said main segment; and
inserting a screw through said first bores to separate and fix said pedicles at said respective distances from said main segment after said segment is cut.

5. A method in accordance with claim 4, wherein said cutting is performed by a cutting device inserted into said bore.

6. A method in accordance with claim 4, wherein said screw comprises a two component screw, each component having coaxial external threads and adapted to rotate separately from the other component.

7. A method in accordance with claim 6, wherein each component has threads of a different pitch.

8. A method in accordance with claim 4, wherein said screw comprises a stenosis screw.

9. A method in accordance with claim 8, wherein said stenosis screw comprises:
a head portion adapted to accept a turning tool for rotating said screw;
a threaded portion adjacent said head portion;
a shoulder portion adjacent the threaded portion; and
a non-threaded pin portion adjacent said shoulder portion.

10. A method in accordance with claim 9, wherein:
the diameter of the first bore corresponds to a diameter of said non-threaded pin portion of said stenosis screw;
a second bore is drilled over the first bore, said second bore having a larger diameter than said first bore and a second length which is shorter than the first length of the first bore, resulting in a two-section bore having a first upper section in said pedicle having a larger diameter than a second lower section in said main segment;
said stenosis screw is inserted into said two-section bore such that:
said unthreaded pin portion penetrates into said lower section of said two-section bore;
said threaded portion penetrates into said bone material of said pedicle surrounding said upper section of said two-section bore;
said shoulder abuts said main segment and limits the penetration of said unthreaded portion into said lower section; and
rotating said screw causes said pedicle to separate from said main segment.

11. A method in accordance with claim 9, wherein one pedicle is separated and fixed at said first respective distance before the other pedicle is separated and fixed at said second respective distance.

12. A method in accordance with claim 10, wherein:
a diameter of said lower section of said two-section bore is approximately 2.7 mm;
a diameter of said upper section of said two-section bore is approximately 4.0 mm;
said first length is approximately 35 mm; and
said second length is approximately 15 mm.

13. A method in accordance with 8, wherein said stenosis screw is one of cylindrical or conical in shape.

14. A method in accordance with claim 4, further comprising:
inserting a drill through a small epidermal incision in the area of the vertebral segment for said drilling; and
inserting a cutting instrument through said bore for said cutting of the vertebral segment.

15. A method in accordance with claim 1, further comprising:
cutting said vertebral segment in a longitudinal direction between the two pedicles in order create a gap between the two pedicles.

16. A method in accordance with claim 15, wherein said gap is approximately 1 mm.

17. A method in accordance with claim 1, wherein said cutting is performed using one of a saw or a shaver-like instrument.

18. A method in accordance with claim 1, wherein each of said first and second distances is approximately 3 to 5 mm.

19. A method in accordance with claim 1, wherein said first and second distances are approximately equal.
